(19) United States
(12) Patent Application Publication
(10) Pub. No.: US 2025/0215041 A1
(43) Pub. Date: Jul. 3, 2025

(54) System and Method for Aqueous Conjugation of Substrates Using Diels-Alder Chemistry

(71) Applicant: Life Technologies Corporation, Carlsbad, CA (US)

(72) Inventors: Dong Li, Santa Clara, CA (US); Amneet Gulati, Cupertino, CA (US)

(21) Appl. No.: 18/989,542

(22) Filed: Dec. 20, 2024

Related U.S. Application Data
(60) Provisional application No. 63/616,326, filed on Dec. 29, 2023.

(51) Int. Cl. C07H 21/00; B01J 19/00

(52) U.S. Cl. CPC C07H 21/00; B01J 19/0046; B01J 2219/0059; B01J 2219/00596; B01J 2219/00722

(57) ABSTRACT

A method for conjugating oligonucleotides to bead supports includes adding 4-(4,6-dimethoxy-1,3,5-triazin-2-yl)-4-methyl-morpholinium to an aqueous suspension including bread supports having carboxy functional groups; adding amino-maleimide to the aqueous suspension; and adding diene modified oligonucleotide to the aqueous suspension.

### CROSS-REFERENCE TO RELATED APPLICATIONS

This application claims the benefit of U.S. Provisional Application No. 63/616,326, filed Dec. 29, 2023, which is incorporated herein by reference in its entirety.

### BACKGROUND

Polymeric particles are increasingly being used as components in separation techniques and to assist with detecting analytes in both chemical and biological systems. For example, polymeric particles have been used in chromatographic techniques to separate target molecules from a solution. In another example, polymeric particles having a magnetic coating are utilized in magnetic separation techniques. More recently, polymeric particles have been used to enhance ELISA-type techniques and can be used to capture proteins or polynucleotides.

In particular, polymer particles conjugated with oligonucleotides can be used to capture complementary polynucleotides. Further, such particles can be used to form multiple copies of the captured polynucleotide and used in sequencing reactions.

However, conventional methods for conjugating oligonucleotides to polymer particles suffer from inefficiencies and stringent process conditions. As such, an improved method for conjugating a polymeric particle would be desirable.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of an example sequencing system.

FIG. 2 includes an illustration of an example system including a sensor array.

FIG. 3 includes an illustration of an example sensor and associated well.

FIG. 4 includes an illustration of an example procedure for forming nucleic acid supports and loading such supports onto a substrate for sequencing.

FIG. 5 includes an illustration of another example procedure for forming nucleic acid supports and loading such supports onto a substrate for sequencing.

FIG. 6 and FIG. 7 illustrate example schema for preparing a bead assembly.

FIG. 8 and FIG. 9 illustrate example schema for preparing a bead support.

The use of the same reference symbols in different drawings indicates similar or identical items.

### DETAILED DESCRIPTION

In an example, a method for conjugating a polymer particle, such as a carboxy functional polymer particle includes activating the carboxy functional group and reacting the activated carboxy functional group with a modified oligonucleotide. For example, the polymer particle can be a carboxy functional polyacrylamide particle. Activating the carboxy functional group can include reacting the carboxy functional group with 4-(4,6-dimethoxy-1,3,5-triazin-2-yl)-4-methylmorpholinium chloride followed by reaction with amino maleimide. The modified oligonucleotide can include a modified 5′ end that includes an amine, a diene, or another reactive species. In particular, the modified oligonucleotide includes a diene or similar moiety at the 5′ end. The reactions can be performed in aqueous solutions.

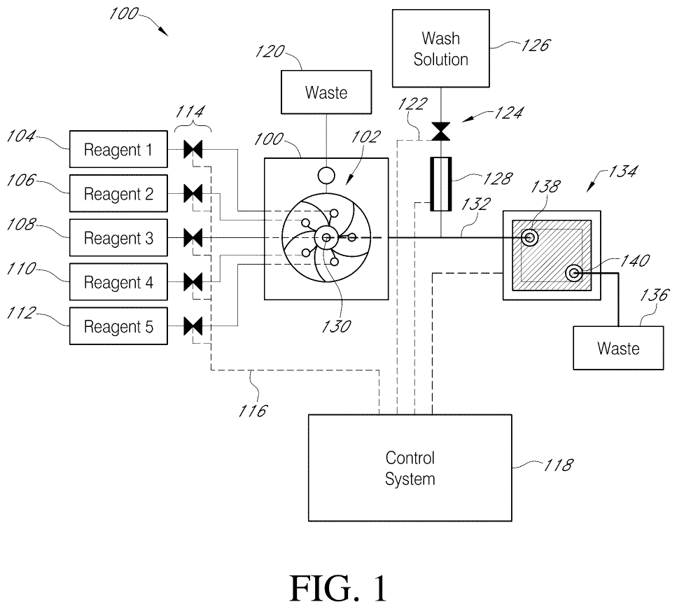

Such oligonucleotide conjugated polymer particles find particular use in sequencing. For example, FIG. 1 diagrammatically illustrates a system for carrying out pH-based nucleic acid sequencing. Each electronic sensor of the apparatus generates an output signal that depends on the value of a reference voltage. The fluid circuit permits multiple reagents to be delivered to the reaction chambers.

In FIG. 1, system 100 containing fluidics circuit 102, which is connected by inlets to at least two reagent reservoirs (104, 106, 108, 110, or 112), to waste reservoir 120, and to biosensor 134 by fluid pathway 132 that connects fluidics node 130 to inlet 138 of biosensor 134 for fluidic communication. Reagents from reservoirs (104, 106, 108, 110, or 112) can be driven to fluidic circuit 102 by a variety of methods including pressure, pumps, such as syringe pumps, gravity feed, and the like, and are selected by control of valves 114. Reagents from the fluidics circuit 102 can be driven through the valves 114 receiving signals from control system 118 to waste container 120. Reagents from the fluidics circuit 102 can also be driven through the biosensor 134 to the waste container 136. The control system 118 includes controllers for valves, which generate signals for opening and closing via electrical connection 116.

The control system 118 also includes controllers for other components of the system, such as wash solution valve 124 connected thereto by electrical connection 122, and reference electrode 128. Control system 118 can also include control and data acquisition functions for biosensor 134. In one mode of operation, fluidic circuit 102 delivers a sequence of selected reagents 1, 2, 3, 4, or 5 to biosensor 134 under programmed control of control system 118, such that in between selected reagent flows, fluidics circuit 102 is primed and washed, and biosensor 134 is washed. Fluids entering biosensor 134 exit through outlet 140 and are deposited in waste container 136 via control of pinch valve regulator. The valve is in fluidic communication with the sensor fluid output 140 of the biosensor 134.

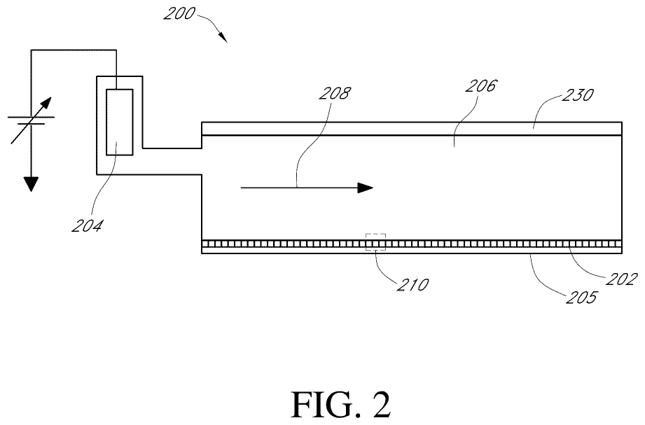

The device including the dielectric layer defining the well formed from the first access and second access and exposing a sensor pad finds particular use in detecting chemical reactions and byproducts, such as detecting the release of hydrogen ions in response to nucleotide incorporation, useful in genetic sequencing, among other applications. In a particular embodiment, a sequencing system includes a flow cell in which a sensory array is disposed, includes communication circuitry in electronic communication with the sensory array, and includes containers and fluid controls in fluidic communication with the flow cell. In an example, FIG. 2 illustrates an expanded and cross-sectional view of a flow cell 200 and illustrates a portion of a flow chamber 206. A reagent flow 208 flows across a surface of a well array 202, in which the reagent flow 208 flows over the open ends of wells of the well array 202. The well array 202 and a sensor array 205 together may form an integrated unit forming a lower wall (or floor) of flow cell 200. A reference electrode 204 may be fluidly coupled to flow chamber 206. Further, a flow cell cover 230 encapsulates flow chamber 206 to contain reagent flow 208 within a confined region.

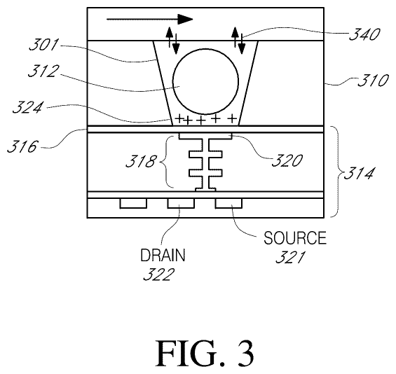

FIG. 3 illustrates an expanded view of a well 301 and a sensor 314, as illustrated at 210 of FIG. 2. The volume, shape, aspect ratio (such as base width-to-well depth ratio), and other dimensional characteristics of the wells may be selected based on the nature of the reaction taking place, as well as the reagents, byproducts, or labeling techniques (if any) that are employed. The sensor 314 can be a chemical field-effect transistor (chemFET), more specifically an ion-sensitive FET (ISFET), with a floating gate 318 having a sensor plate 320 optionally separated from the well interior by a passivation layer 316. The sensor 314 can be responsive to (and generate an output signal related to) the amount of a charge 324 present on passivation layer 316 opposite the sensor plate 320. Changes in the charge 324 can cause changes in a current between a source 321 and a drain 322 of the chemFET. In turn, the chemFET can be used directly to provide a current-based output signal or indirectly with additional circuitry to provide a voltage-based output signal. Reactants, wash solutions, and other reagents may move in and out of the wells by a diffusion mechanism 340.

In an embodiment, reactions carried out in the well 301 can be analytical reactions to identify or determine characteristics or properties of an analyte of interest. Such reactions can generate directly or indirectly byproducts that affect the amount of charge adjacent to the sensor plate 320. If such byproducts are produced in small amounts or rapidly decay or react with other constituents, then multiple copies of the same analyte may be analyzed in the well 301 at the same time in order to increase the output signal generated. In an embodiment, multiple copies of an analyte may be attached to a solid phase support 312, either before or after deposition into the well 301. The solid phase support 312 may be microparticles, nanoparticles, beads, solid or porous comprising gels, or the like. For simplicity and ease of explanation, solid phase support 312 is also referred herein as a particle or bead. For a nucleic acid analyte, multiple, connected copies may be made by rolling circle amplification (RCA), exponential RCA, or like techniques, to produce an amplicon without the need of a solid support.

The well wall structure 310 can be formed of one or more layers of material. In an example, the well wall structure 310 can have a thickness (t) extending from the lower surface to the upper surface in a range of 0.3 micrometers to 10 micrometers, such as a range of 0.5 micrometers to 6 micrometers. The wells 301 can have a characteristic diameter, defined as the square root of 4 times the cross-sectional area (A) divided by Pi (e.g., sqrt (4*A/π), of not greater than 5 micrometers, such as not greater than 3.5 micrometers, not greater than 2.0 micrometers, not greater than 1.6 micrometers, not greater than 1.0 micrometers, not greater than 0.8 micrometers or even not greater than 0.6 micrometers. In an example, the characteristic diameter is at least 0.01 micrometers.

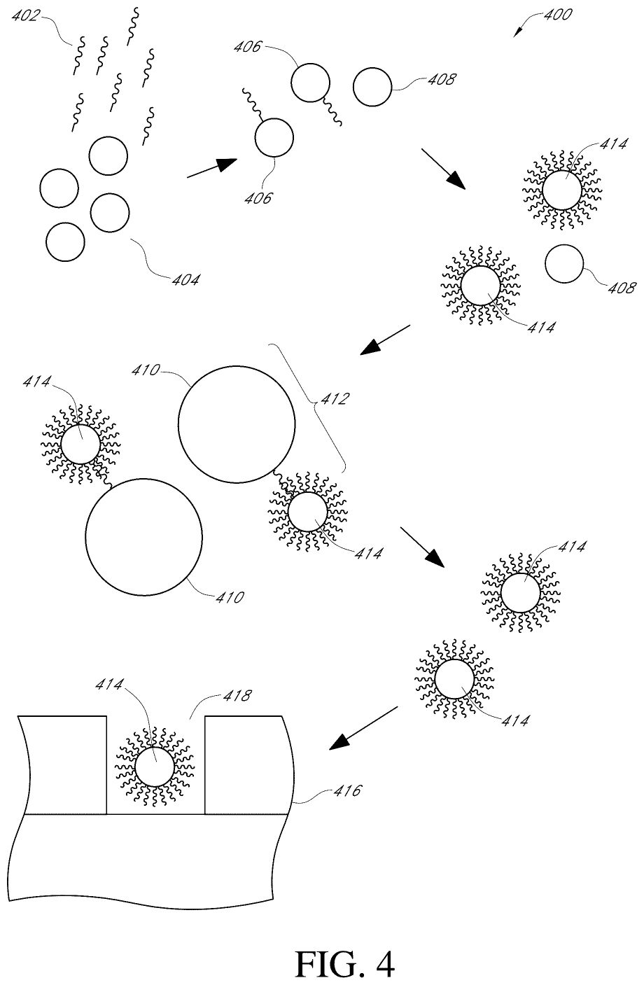

FIG. 4 includes an illustration of an example method for forming nucleic acid supports and loading such supports onto a substrate for sequencing. A plurality of target nucleic acids 402 and supports 404 are provided in solution. Some of the target nucleic acids bind with the supports 406 while other supports 408 remain free of target nucleic acids. Target nucleic acids can be derived from one or more DNA or RNA samples, such as genomic DNA from different subjects. The target nucleic acids from each sample can be combined and undergo the process described below. Alternatively, the target nucleic acids from each sample can be processed separately.

The target nucleic acid secured to the supports 406 are amplified to form multiple copies of the target nucleic acids on the supports 414. Supports 408 remain free of target nucleic acids. Amplification can include a polymerase chain reaction (PCR) amplification, such as emulsion PCR, or isothermal amplification in an emulsion or in bulk solution, such as recombinase-polymerase amplification (RPA).

Capture primers having a capture moiety and complementary to terminal ends of the target nucleic acids secured to the supports can be used to bind the supports 414 to magnetic beads 410 having a moiety that binds with the capture moiety. In an example capture moiety is biotin which binds to streptavidin, for example. As illustrated, the supports 414 including target nucleic acids combined with magnetic beads 410 to form a complex 412. A magnetic field can be used to secure the magnetic beads 410 and the supports 414 including target nucleic acids while the remaining solution is washed away, including supports 408 free of target nucleic acid.

The supports 414 including target nucleic acids can be separated from the magnetic beads 410 by denaturing or de-hybridizing the capture moiety from the target nucleic acid. For example, a change in ion concentration or an increase in temperature may result in the release of the capture primer from the target nucleic acid. Using a magnetic field, the magnetic beads 410 can be separated from the supports 414 including target nucleic acids.

The supports 414 including the target nucleic acid can be applied to the substrate such as substrate 416. In an example, the substrate includes a set of wells 418 into which the supports 414 are deposited. Sequencing can be performed using the supports including target nucleic acid, for example by sequencing-by-synthesis.

In an example, a sequencing primer can be added to the wells 418 or the bead support 414 can be pre-exposed to the primer prior to placement in the well 418. In particular, the bead support 414 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 418 to facilitate detectible reactions, such as nucleotide incorporation.

Sequencing can be performed by detecting nucleotide addition. Nucleotide addition can be detected using methods such as fluorescent emission methods or ion detection methods. For example, a set of fluorescently labeled nucleotides can be provided to the system 416 and can migrate to the well 418. Excitation energy can be also provided to the well 418. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 418 can change. Such a change in pH can be detected by ion sensitive field effect transistors (ISFET). As such, a change in pH can be used to generate a signal indicating the order of nucleotides complementary to the polynucleotide of the particle 410.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 10², 10³, 10⁴, 10⁵, 10⁶, 10⁷ or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 4, in another example, a well 418 of the array of wells can be operatively connected to measuring devices. For example, for fluorescent emission methods, a well 418 can be operatively coupled to a light detection device. In the case of ionic detection, the lower surface of the well 418 may be disposed over a sensor pad of an ionic sensor, such as a field effect transistor.

One example system involving sequencing via detection of ionic byproducts of nucleotide incorporation is the Ion Torrent PGM™, Proton™ or S5™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, S5™, or Genexus™ sequencer detects the nucleotide incorporations by detecting the hydrogen ion byproducts of the nucleotide incorporations. The Ion Torrent PGM™, Proton™, S5™, or Genexus™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

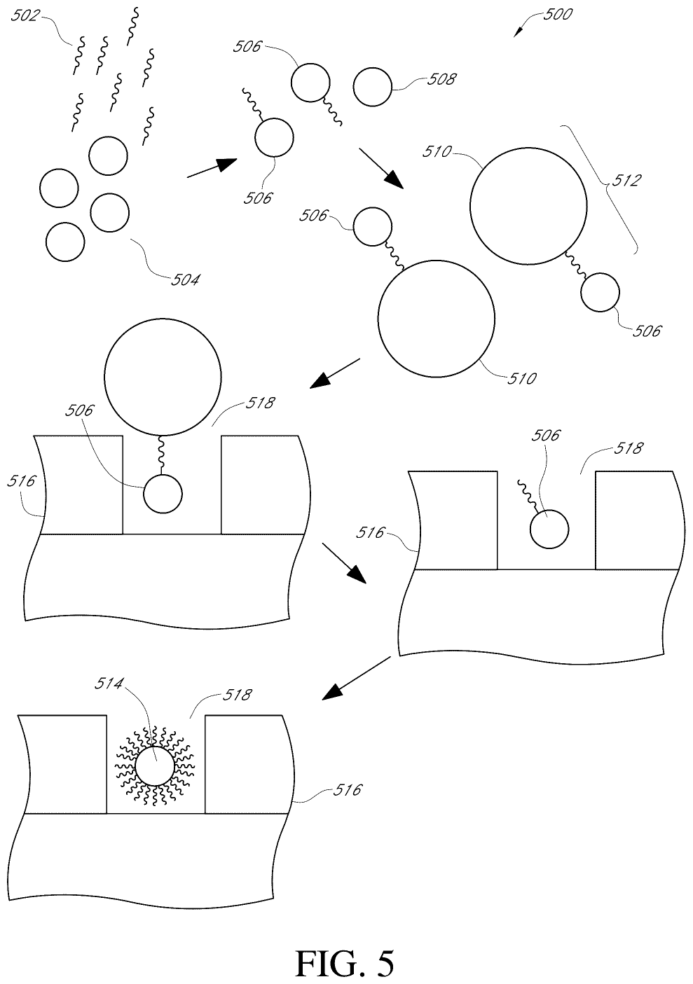

In particular, the solid phase support, such a bead support, can include copies of polynucleotides. In a particular example illustrated in FIG. 5, polymeric particles can be used as a support for polynucleotides during sequencing techniques. For example, such hydrophilic particles can immobilize a polynucleotide for sequencing using fluorescent sequencing techniques. In another example, the hydrophilic particles can immobilize a plurality of copies of a polynucleotide for sequencing using ion-sensing techniques. Alternatively, the above described treatments can improve polymer matrix bonding to a surface of a sensor array. The polymer matrices can capture analytes, such as polynucleotides for sequencing.

A bead support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Supports may be porous or non-porous and may have swelling or non-swelling characteristics. In some embodiments, a support is an Ion Sphere Particle. Example bead supports are disclosed in U.S. Pat. No. 9,243,085, titled “Hydrophilic Polymeric Particles and Methods for Making and Using Same,” and in U.S. Pat. No. 9,868,826, titled “Polymer Substrates Formed from Carboxy Functional Acrylamide,” each of which is incorporated herein by reference.

In some embodiments, the solid support is a “microparticle,” “bead,” “microbead,” etc., (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). In an example, the support is at least 0.1 microns. Microparticles or bead supports may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. Magnetization can facilitate collection and concentration of the microparticle-attached reagents (e.g., polynucleotides or ligases) after amplification, and can also facilitate additional steps (e.g., washes, reagent removal, etc.). In certain embodiments, a population of microparticles having different shapes sizes or colors is used. The microparticles can optionally be encoded, e.g., with quantum dots such that each microparticle or group of microparticles can be individually or uniquely identified.

Magnetic beads (e.g., Dynabeads from Dynal, Oslo, Norway) can have a size in a range of 1 micron to 100 microns, such as 2 microns to 100 microns. The magnetic beads can be formed of inorganic or organic materials including, but not limited to, glass (e.g., controlled pore glass), silica, zirconia, cross-linked polystyrene, polystyrene, or a combination thereof.

In some embodiments, a bead support is functionalized for attaching a population of first primers. In some embodiments, a bead is any size that can fit into a reaction chamber. For example, one bead can fit in a reaction chamber. In some embodiments, more than one bead fit in a reaction chamber. In some embodiments, the smallest cross-sectional length of a bead (e.g., diameter) is about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In general, the bead support can be treated to include a biomolecule, including nucleosides, nucleotides, nucleic acids (oligonucleotides and polynucleotides), polypeptides, saccharides, polysaccharides, lipids, or derivatives or analogs thereof. For example, a polymeric particle can bind or attach to a biomolecule. A terminal end or any internal portion of a biomolecule can bind or attach to a polymeric particle. A polymeric particle can bind or attach to a biomolecule using linking chemistries. A linking chemistry includes covalent or non-covalent bonds, including an ionic bond, hydrogen bond, affinity bond, dipole-dipole bond, van der Waals bond, and hydrophobic bond. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

As illustrated in FIG. 5, a plurality of bead supports 504 can be placed in a solution along with a plurality of polynucleotides 502 (target or template polynucleotides). Target or template polynucleotides can be derived from one or more DNA or RNA samples, such as genomic DNA from different subjects. The target or template polynucleotides from each sample can be combined and undergo the process described below. Alternatively, the target or template polynucleotides from each sample can be processed separately.

The plurality of bead supports 504 can be activated or otherwise prepared to bind with the polynucleotides 502. For example, the bead supports 504 can include an oligonucleotide (capture primer) complementary to a portion of a polynucleotide of the plurality of polynucleotides 502. In another example, the bead supports 504 can be modified with target polynucleotides 502 using techniques such as biotin-streptavidin binding.

In some embodiments, the template nucleic acid molecules (template polynucleotides or target polynucleotides) can be derived from a sample that can be from a natural or non-natural source. The nucleic acid molecules in the sample can be derived from a living organism or a cell. Any nucleic acid molecule can be used, for example, the sample can include genomic DNA covering a portion of or an entire genome, mRNA, or miRNA from the living organism or cell. In other embodiments, the template nucleic acid molecules can be synthetic or recombinant. In some embodiments, the sample contains nucleic acid molecules having substantially identical sequences or having a mixture of different sequences. Illustrative embodiments are typically performed using nucleic acid molecules that were generated within and by a living cell. Such nucleic acid molecules are typically isolated directly from a natural source such as a cell or a bodily fluid without any in vitro amplification. Accordingly, the sample nucleic acid molecules are used directly in subsequent steps. In some embodiments, the nucleic acid molecules in the sample can include two or more nucleic acid molecules with different sequences.

The methods can optionally include a target enrichment step before, during, or after the library preparation and before a pre-seeding reaction. Target nucleic acid molecules, including target loci or regions of interest, can be enriched, for example, through multiplex nucleic acid amplification or hybridization. A variety of methods can be used to perform multiplex nucleic acid amplification to generate amplicons, such as multiplex PCR, and can be used in an embodiment. Enrichment by any method can be followed by a universal amplification reaction before the template nucleic acid molecules are added to a pre-seeding reaction mixture. Any of the embodiments of the present teachings can include enriching a plurality of at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 target nucleic acid molecules, target loci, or regions of interest. In any of the disclosed embodiments, the target loci or regions of interest can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the template nucleic acid molecule. In other embodiments, the target loci or regions of interest can be between about 1 and 10,000 nucleotides in length, for example between about 2 and 5,000 nucleotides, between about 2 and 3,000 nucleotides, or between about 2 and 2,000 nucleotides in length. In any of the embodiments of the present teachings, the multiplex nucleic acid amplification can include generating at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 copies of each target nucleic acid molecule, target locus, or region of interest.

In some embodiments, after the library preparation and optional enrichment step, the library of template nucleic acid molecules can be templated onto one or more supports. The one or more supports can be templated in two reactions, a seeding reaction to generate pre-seeded solid supports and a templating reaction using the one or more pre-seeded supports to further amplify the attached template nucleic acid molecules. The pre-seeding reaction is typically an amplification reaction and can be performed using a variety of methods. For example, the pre-seeding reaction can be performed in an RPA reaction, a template walking reaction, or a PCR. In an RPA reaction, template nucleic acid molecules are amplified using a recombinase, polymerase, and optionally a recombinase accessory protein in the presence of primers and nucleotides. The recombinase and optionally the recombinase accessory protein can dissociate at least a portion of a double stranded template nucleic acid molecules to allow primers to hybridize that the polymerase can then bind to initiate replication. In some embodiments, the recombinase accessory protein can be a single-stranded binding protein (SSB) that prevents the re-hybridization of dissociated template nucleic acid molecules. Typically, RPA reactions can be performed at isothermal temperatures. In a template walking reaction, template nucleic acid molecules are amplified using a polymerase in the presence of primers and nucleotides in reaction conditions that allow at least a portion of double-stranded template nucleic acid molecules to dissociate such that primers can hybridize, and the polymerase can then bind to initiate replication. In PCR, the double-stranded template nucleic acid molecules are dissociated by thermal cycling. After cooling, primers bind to complementary sequences and can be used for replication by the polymerase. In any of the aspects of the present teachings, the pre-seeding reaction can be performed in a pre-seeding reaction mixture, which is formed with the components necessary for amplification of the template nucleic acid molecules. In any of the disclosed aspects, the pre-seeding reaction mixture can include some or all of the following: a population of template nucleic acid molecules, a polymerase, one or more solid supports with a population of attached first primers, nucleotides, and a cofactor such as a divalent cation. In some embodiments, the pre-seeding reaction mixture can further include a second primer and optionally a diffusion-limiting agent. In some embodiments, the population of template nucleic acid molecules comprise template nucleic acid molecules joined to at least one adaptor sequence which can hybridize to the first or second primers. In some embodiments, the reaction mixture can form an emulsion, as in emulsion RPA or emulsion PCR. In pre-seeding reactions carried out by RPA reactions, the pre-seeding reaction mixture can include a recombinase and optionally a recombinase accessory protein. The various components of the reaction mixture are discussed in further detail herein.

In a particular embodiment of seeding, the hydrophilic particles and polynucleotides are subjected to polymerase chain reaction (PCR) amplification or recombinase polymerase amplification (RPA). In an example, the particles 504 include a capture primer complementary to a portion of the template polynucleotide 502. The template polynucleotide can hybridize to the capture primer. The capture primer can be extended to form beads 506 that include a target polynucleotide attached thereto. Other beads may remain unattached to a target nucleic acid and other template polynucleotide can be free floating in solution.

In an example, the bead support 506 including a target polynucleotide can be attached to a magnetic bead 510 to form a bead assembly 512. In particular, the magnetic bead 510 is attached to the bead support 506 by a double stranded polynucleotide linkage. In an example, a further probe including a linker moiety can hybridize to a portion of the target polynucleotide on the bead support 506. The linker moiety can be attached to a complementary linker moiety on the magnetic bead 510. In another example, the template polynucleotide used to form the target nucleic acid attached to beads 506 can include a linker moiety that attaches to the magnetic bead 510. In another example, the template polynucleotide complementary to target polynucleotide attached to the bead support 506 can be generated from a primer that is modified with a linker that attaches to the magnetic bead 510.

The linker moiety attached to the polynucleotide and the linker moiety attached to the magnetic bead can be complementary to and attach to each other. In an example, the linker moieties have affinity and can include: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In a particular example, the linker moiety attached to the polynucleotide includes biotin and the linker moiety attached to the magnetic bead includes streptavidin.

The bead assemblies 512 can be applied over a substrate 516 of a sequencing device that includes wells 518. In an example, a magnetic field can be applied to the substrate 516 to draw the magnetic beads 510 of the bead assembly 512 towards the wells 518. The bead support 506 enters the well 518. For example, a magnet can be moved in parallel to a surface of the substrate 516 resulting in the deposition of the bead support 406 in the wells 518.

The bead assembly 512 can be denatured to remove the magnetic bead 510 leaving the bead support 506 in the well 518. For example, hybridized double-stranded DNA of the bead assembly 512 can be denatured using thermal cycling or ionic solutions to release the magnetic bead 510 and template polynucleotides having a linker moiety attached to the magnetic bead 510. For example, the double-stranded DNA can be treated with low ion-content aqueous solutions, such as deionized water, to denature and separate the strands. In an example, a foam wash can be used to remove the magnetic beads.

Optionally, the target polynucleotides 506 can be amplified, referred to herein as templating, while in the well 518, to provide a bead support 514 with multiple copies of the target polynucleotides. In particular, the bead 514 has a monoclonal population of target polynucleotides. Such an amplification reactions can be performed using polymerase chain reaction (PCR) amplification, recombination polymerase amplification (RPA) or a combination thereof. Alternatively, amplification can be performed prior to depositing the bead support 514 in the well.

In a particular embodiment, an enzyme such as a polymerase is present, bound to, or is in close proximity to the particles or beads. In an example, a polymerase is present in solution or in the well to facilitate duplication of the polynucleotide. A variety of nucleic acid polymerase may be used in the methods described herein. In an example embodiment, the polymerase can include an enzyme, fragment, or subunit thereof, which can catalyze duplication of the polynucleotide. In another embodiment, the polymerase can be a naturally occurring polymerase, recombinant polymerase, mutant polymerase, variant polymerase, fusion or otherwise engineered polymerase, chemically modified polymerase, synthetic molecules, or analog, derivative, or fragment thereof. Example enzymes, solutions, compositions, and amplification methods can be found in WO2019/094,524, titled “METHODS AND COMPOSITIONS FOR MANIPULATING NUCLEIC ACIDS”, which is incorporated herein by reference in its entirety.

While the polynucleotides of bead support 514 are illustrated as being on a surface, the polynucleotides can extend within the bead support 514. Hydrogel and hydrophilic particles having a low concentration of polymer relative to water can include polynucleotide segments on the interior of and throughout the bead support 514 or polynucleotides can reside in pores and other openings. In particular, the bead support 514 can permit diffusion of enzymes, nucleotides, primers, and reaction products used to monitor the reaction. A high number of polynucleotides per particle produces a better signal.

In an example embodiment, the bead support 514 can be utilized in a sequencing device. For example, a sequencing device 516 can include an array of wells 518.

In an example, a sequencing primer can be added to the wells 518 or the bead support 514 can be pre-exposed to the primer prior to placement in the well 518. In particular, the bead support 514 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 518 to facilitate detectible reactions, such as nucleotide incorporation.

Sequencing can be performed by detecting nucleotide addition. Nucleotide addition can be detected using methods such as fluorescent emission methods or ion detection methods. For example, a set of fluorescently labeled nucleotides can be provided to the system 516 and can migrate to the well 518. Excitation energy can be also provided to the well 518. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 518 can change. Such a change in pH can be detected by ion sensitive field effect transistors (ISFET). As such, a change in pH can be used to generate a signal indicating the order of nucleotides complementary to the polynucleotide of the particle 510.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 10², 10³, 10⁴, 10⁵, 10⁶, 10⁷ or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 5, in another example, a well 518 of the array of wells can be operatively connected to measuring devices. For example, for fluorescent emission methods, a well 518 can be operatively coupled to a light detection device. In the case of ionic detection, the lower surface of the well 418 may be disposed over a sensor pad of an ionic sensor, such as a field effect transistor.

One example system involving sequencing via detection of ionic byproducts of nucleotide incorporation is the Ion Torrent PGM™, Proton™, S5™, or Genexus™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, S5™, or Genexus™ sequencer detects the nucleotide incorporations by detecting the hydrogen ion byproducts of the nucleotide incorporations. The Ion Torrent PGM™, Proton™, S5™, or Genexus™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

The oligonucleotide conjugated polymer particles can be used in a process to extend the oligonucleotide complementary to a template nucleotide. The template is complementary to a target nucleic acid. As a result, a sequence of the target nucleic acid is bound to the bead. The target nucleic acid can be amplified or templated on to the oligonucleotide conjugated polymer particle to form a polymer particle having a monoclonal population of target nucleic acids bound thereto.

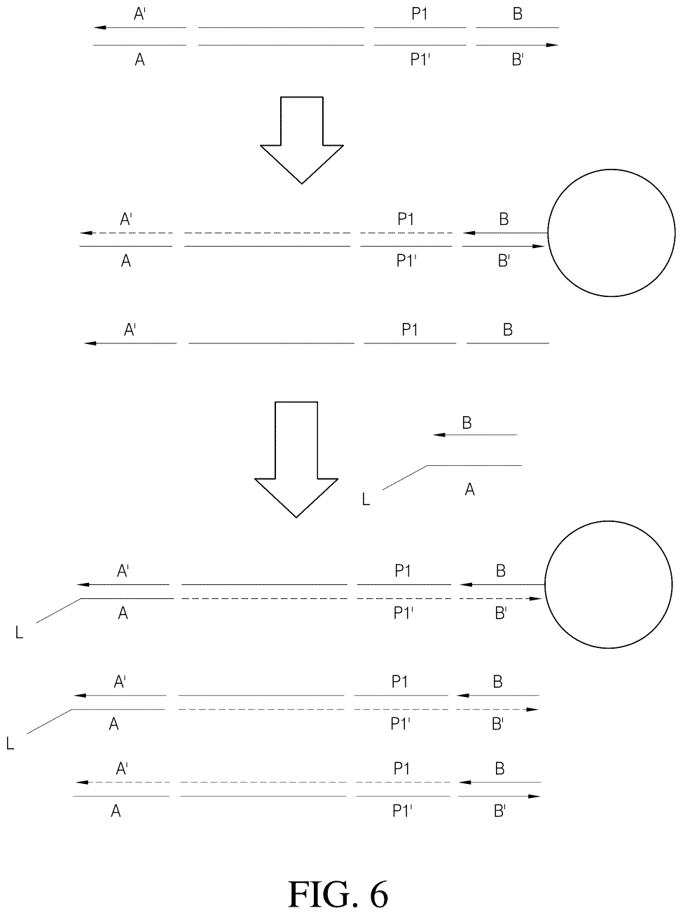

In an example illustrated in FIG. 6, a target polynucleotide B-A′ and its complement, a template polynucleotide (A-B′), are amplified in the presence of a bead support having a capture primer. The target polynucleotide has a capture portion (B) the same as or substantially similar to a sequence of the capture primer coupled to the bead support. Substantially similar sequences are sequences whose complements can hybridize to each of the substantially similar sequences. The bead support can have a capture primer that is the same sequence or a sequence substantially similar to that of the B portion of the target polynucleotide to permit hybridization of the complement of the capture portion (B) of the target polynucleotide with the capture primer attached to the bead support. Optionally, the target polynucleotide can include a second primer location (P1) adjacent to the capture portion (B) of the target polynucleotide and can further include a target region adjacent the primers and bounded by complement portion (A′) to a sequencing primer portion (A) of the target polynucleotide.

When amplified in the presence of the bead support including a capture primer, the template polynucleotide complementary to the target polynucleotide can hybridize with the capture primer (B). The target polynucleotide can remain in solution. The system cannot undergo an extension in which the capture primer B is extended complementary to the template polynucleotide yielding a target sequence bound to the bead support.

A further amplification can be performed in the presence of a free primer (B), the bead support, and a free modified sequencing primer (A) a having a linker moiety (L) attached thereto. The primer (B) and the modified primer (L-A) can interfere with the free-floating target polynucleotide and template polynucleotide, hindering them from binding to the bead support and each other. In particular, the modified sequencing primer (A) having the linker moiety attached thereto can hybridize with the complementary portion (A′) of the target polynucleotide attached to the bead support. Optionally, the linker modified sequencing primer L-A hybridized to the target polynucleotide can be extended forming a linker modified template polynucleotide. Such linker modified template polynucleotide hybridize to the target nucleic acid attached to the bead support can then be captured by a magnetic bead and used for magnetic loading of the sequencing device.

The amplification or extensions can be performed using polymerase chain reaction (PCR) amplification, recombinase polymerase amplification (RPA), or other amplification techniques. In a particular example, each step of the scheme illustrated in FIG. 6 is performed using PCR amplification.

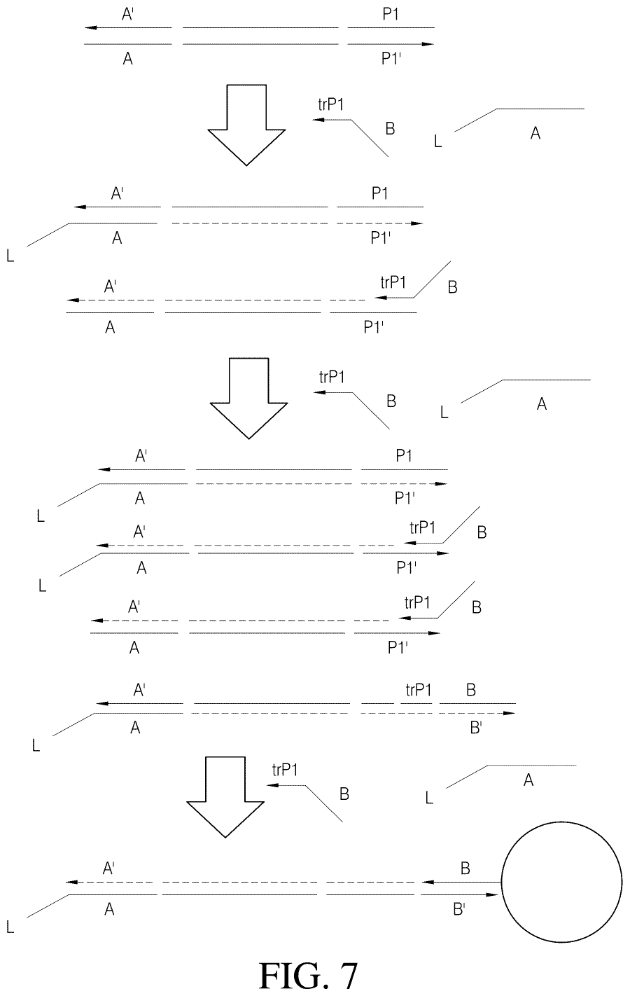

In another example illustrated in FIG. 7, an alternative scheme includes a target polynucleotide (P1-A′) and its complement template polynucleotide (A-P1′). The target polynucleotide and template polynucleotide are amplified in a solution including a linker modified sequencing primer (L-A) and a truncated P1 primer (trP1) having a portion having the sequence of the capture primer (B). In an example, the truncated P1 primer (trP1) includes a subset of the sequence of P1 or all of the sequence P1. During subsequent amplifications in the presence of the linker modified sequencing primer (L-A) and truncated P1 primer (trP1-B), a single species includes a linker modified template polynucleotide (L-A-B′) operable to hybridize with a bead support having a capture primer (B). Accordingly, the linker modified template polynucleotide (L-A-B′) hybridizes with the capture primer (B) on the bead and is extended to form a target polynucleotide (B-A′) attached to the bead support.

The linker modified template polynucleotide hybridize to the target polynucleotide attached bead can be utilized to attached to a magnetic bead, which can be used to implement magnetic loading of the bead into a sequencing device. As described above, the linker moiety of the linker modified template polynucleotide can take various forms, such as biotin, which can bind to linker moieties attached to the magnetic bead, such as streptavidin. Each of the amplification reactions can be undertaken using PCR, RPA, or other amplification techniques. In the example illustrated in FIG. 7, the scheme can be implemented using three cycles of polymerase chain reaction (PCR). Such a series of PCR reactions results in a greater percentage of bead supports having a single target polynucleotide attached thereto. As a result, more monoclonal populations can be generated in wells in the sequencing device.

An example polymer particle includes a polyacrylamide polymer particle with carboxy functionality. An example particle is described in WO 2017/004556, titled “POLYMER SUBSTRATES FORMED FROM CARBOXY FUNCTIONAL ACRYLAMIDE.”

In an exemplary embodiment, a polymer substrate, such as a polymer particle, is formed from a carboxyl functional monomer. In an example, the carboxyl functional monomer has a protection group in place of the OH of the carboxyl group. The protection group can protect the OH group during the polymerization reaction or can render the monomer more miscible with hydrophobic phases. Once the monomer is polymerized, the protection group can be removed, providing a polymer network with carboxyl functional sites. Such sites can be used to attach functionality to the polymer substrate, such as oligomer primers.

In a particular example, a monomer solution can be distributed to a dispersed hydrophobic phase within a hydrophilic or aqueous continuous phase. In an example, the dispersed hydrophobic phase can be formed from a hydrophobic polymer bead. The monomer solution can include a protected carboxyl functional monomer, such as a protected carboxyl functional acrylamide. Optionally, the monomer solution can further include other monomers, crosslinkers, porogens, catalysts, or any combination thereof.

In an example, the monomer can include a protected carboxyl functional acrylamide monomer. In particular, the protected carboxyl functional acrylamide includes a protection group protecting the hydrophilic OH of the carboxyl functionality. The protection group can protect the OH group, preventing reaction during polymerization or rendering the monomer more miscible with hydrophobic phases. In particular, the protection group is cleavable from the monomer or from a polymer network formed from the monomer. For example, the protection group can be acid cleavable, in particular, at a pH that does not cause hydrolysis of the polymer network.

For example, the protection group can include a silyl group. In another example, the protection group can include a linear or branched alkyl group having at least three carbons. For example, the alkyl group can include 3 to 8 carbons, such as 3 to 6 carbons or 3 to 5 carbons. In particular, the protection group can be a branched alkyl group, such as a branched alkyl group having between 3 and 5 carbons, such as 4 carbons. For example, the monomer can have the formula (I):

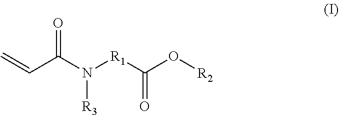

wherein R₁ is an alkyl group having between 3 and 10 carbons, is a polyether group having between 1 and 10 ether units, or is another non-ionic polar group, wherein R₂ is a linear or branched alkyl group having between 3 and 8 carbons or is a silyl group, and wherein R₃ is hydrogen or an alkyl group having between 1 and 6 carbons. In a particular example, R₁ is an alkyl group having between 3 and 10 carbons or is a polyether group having between 1 and 10 ether units. For example, R₁ can be an alkyl group having 3 to 6 carbons, such as 3 to 5 carbons. In another example, R₁ can be a polyether group including units, such as including ethylene oxide or propylene oxide units, in a range of 2 to 6 units, such as 2 to 4 units. In a further example, R₁ can be a non-ionic polar group, for example, including an amide. In an example, R₂ is a branched alkyl group, for example, having 3 to 5 carbons, such as 4 carbons. In particular, R₂ can be an isopropyl, isobutyl, sec-butyl, or tert-butyl group, or any combination thereof. The silyl group can be a trialkyl silyl group, an organo disilyl group, or an organo trisilyl group. For example, the trialkyl silyl group can be a trimethyl silyl or a triethyl silyl group. In a further example, R₃ is hydrogen. In another example, R₃ is a methyl or ethyl group.

In an example, the monomer can have the formula (II):

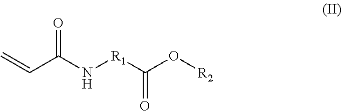

wherein R₁ is an alkyl group having between 3 and 10 carbons or is a polyether group having between 1 and 10 ether units, and wherein R₂ is a linear or branched alkyl group having between 3 and 8 carbons or is a silyl group. For example, R₁ can be an alkyl group having 3 to 6 carbons, such as 3 to 5 carbons. In another example, R₁ can be a polyether group including units, such as including ethylene oxide or propylene oxide units, in a range of 2 to 6 units, such as 2 to 4 units. In an example, R₂ is a branched alkyl group, for example, having 3 to 5 carbons, such as 4 carbons. In particular, R₂ can be an isopropyl, isobutyl, sec-butyl, or tert-butyl group, or any combination thereof. The silyl group can be a trialkyl silyl group, an organo disilyl group, or an organo trisilyl group. For example, the trialkyl silyl group can be a trimethyl silyl or a triethyl silyl group.

In a particular example, the protected carboxyl functional monomer can be acrylamidobutanoate protected with a tert-butyl protection group and having the formula (III):

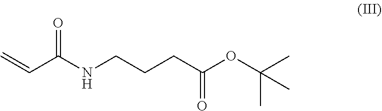

In an example, the protected carboxyl functional monomers of formulas (I), (II), or (III) can be formed by reacting a protected amino alkanoic acid hydrochloride, such as an amino alkanoic alkyl ester hydrocholoride, with acryloyl chloride. For example, stoichiometric quantities of an amino alkanoic alkyl ester hydrocholoride, such as aminobutyric acid t-butyl ester hydrochloride, in a dichloromethane solvent can be mixed with a potassium carbonate solution in water at a temperature in a range of −10° C. to 10° C., such as −5° C. to 5° C. An acryloyl chloride solution can be added and the mixture stirred under the same thermal conditions. The mixture can be extracted with a solvent, such as dichloromethane. The solvent can be removed under reduced pressure or vacuum.

In an example, the monomer described above can be polymerized to form a polymer substrate. For example, the polymer substrate can be a polymer coating or film. In another example, the polymer substrate can be a polymer particle or bead. For example, a polymer particle can be formed using emulsion polymerization or can be formed in a dispersed hydrophobic phase within a hydrophilic continuous phase.

In an example, the polymeric particle can be converted to a hydrophilic polymeric particle by removing at least a portion of the hydrophobic protection groups. For example, the hydrophobic protection groups can be acid-cleaved from the polymeric particles. In particular, such removing can remove substantially all of the hydrophobic protection groups from the polymeric particle, such as removing at least 80% of the hydrophobic protection groups, or even at least 90% of the hydrophobic protection groups.

In an example, the hydrophobic protection groups are acid-cleaved through the addition of an acid, such as an organic acid. In particular, the organic acid can have a pKa in a range of 3.0 to 5.5. For example, the organic acid can include acetic acid, lactic acid, citric acid, or any combination thereof. Alternatively, inorganic acids can be used. For example, a sulfuric acid solution can be used.

Once at least a portion of the hydrophobic protection groups is removed, a hydrophilic particle is formed. The hydrophilic particle includes carboxyl functionality. In an example, the hydrophilic particle 112 can be a hydrogel particle including a hydrogel polymer. A hydrogel is a polymer that can absorb at least 20% of its weight in water, such as at least 45%, at least 65%, at least 85%, at least 100%, at least 300%, at least 1000%, at least 1500%, or even at least 2000% of its weight in water, but not greater than 106%.

Biomolecules, such as oligonucleotides or proteins, can be conjugated to the carboxy functional polymers, such as those described above. In particular, the carboxy functional groups can be activated to facilitate conjugation to the biomolecules.

In an example, the carboxy functional groups can be activated using 4-(4,6-dimethoxy-1,3,5-triazin-2-yl)-4-methylmorpholinium chloride (DMTMM) and amino-maleimide to form functionality that can react with a modified oligonucleotide.

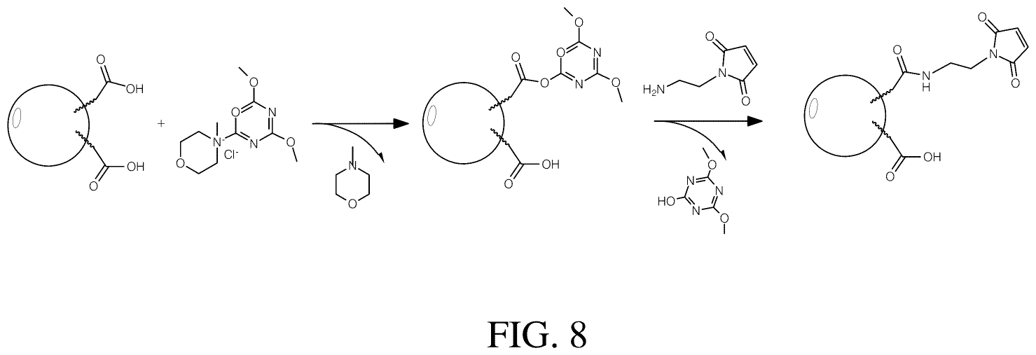

For example, as illustrated in FIG. 8, DMTMM can be added to a suspension of carboxy functional beads. The suspension can be an aqueous suspension and optionally include other solvents and buffer systems.

In an example, the DMTMM can be dissolved in a solvent prior to adding the DMTMM to the suspension. Example solvents include an amide, a urea, a carbonate, an ether, a sulfoxide, a sulfone, a hindered alcohol, or a combination thereof. An exemplary amide or urea includes formamide, N,N-dimethylformamide, acetamide, N,N-dimethylacetamide, hexamethylphosphoramide, pyrrolidone, N-methylpyrrolidone, N,N,N′,N′-tetramethylurea, N,N′-dimethyl-N,N′-trimethyleneurea, or a combination thereof. An exemplary carbonate includes dimethyl carbonate, propylene carbonate, or a combination thereof. An exemplary ether includes tetrahydrofuran. An exemplary sulfoxide or sulfone includes dimethylsulfoxide, dimethylsulfone, or a combination thereof. An exemplary hindered alcohol includes tert-butyl alcohol. In a particular example, the solvent includes N-methylpyrrolidone. In another example, the solvent includes dimethylformamide. In a further example, the solvent includes dimethylsulfoxide.

Example buffer systems include phosphate-buffered saline (PBS) buffer or TE buffer systems. Optionally, the suspension can include a surfactant, such as TWEEN® 20.

The DMTMM can be added to an equivalence ratio relative to the number of carboxy functional groups in a range of 1 eq. to 150 eq., such as 2 eq. to 100 eq., 8 eq. to 75 eq., or 20 eq. to 60 eq. In addition to the DMTMM, a base can be added to the reaction mixture. Example bases include sodium hydroxide or amines, for example, hindered amines, such as triethylamine or diisopropylethylamine (DIPEA).

The reaction mixture can be held at a temperature in a range of 0° C. to 35° C., such as a range of 5° C. to 28° C., or a range of 20° C. to 25° C. The reaction mixture can be incubated for a period in a range of 10 minutes to 100 hours, such as a range of 10 minutes to 60 hours, a range of 30 minutes to 16 hours, or a range of 1 hour to 3 hours. The carboxy groups are converted to a functional group incorporating the methyl-morpholinium and dimethoxy triazine is released, as illustrated in FIG. 8.

An amino-maleimide is added to the suspension. The amino-maleimide can be an amino alkyl maleimide, aminophenyl maleimide, or similar amino functionalized maleimide. For example, the amino-maleimide can be an amino alkyl maleimide, such as aminoethyl maleimide, aminopropyl maleimide, or aminobutyl maleimide. In a particular example, the amino-maleimide is aminoethyl maleimide, such as N-(2-aminoethyl) maleimide.

For example, the amino-maleimide can be added to an equivalent relative to the starting number of carboxy functional groups of in a range of 1 eq. to 150 eq., such as 2 eq. to 100 eq., 8 eq. to 75 eq., or 20 eq. to 60 eq.

The reaction mixture can be incubated at a temperature in a range of 0° C. to 35° C., such as a range of 5° C. to 28° C., or a range of 20° C. to 25° C. The reaction mixture can be incubated for a period in a range of 10 minutes to 100 hours, such as a range of 10 minutes to 60 hours, a range of 30 minutes to 16 hours, or a range of 1 hour to 3 hours.

As a result, the carboxy functional group is converted to an amide group with of the amino-maleimide. Such functionality can be conjugated to a diene modified oligonucleotide.

Alternatively, the DMTMM and amino-maleimide can be added concurrently to the solution. The reaction mixture can be held at a temperature in a range of 0° C. to 35° C., such as a range of 5° C. to 28° C., or a range of 20° C. to 25° C. The reaction mixture can be incubated for a period in a range of 10 minutes to 100 hours, such as a range of 10 minutes to 60 hours, a range of 30 minutes to 16 hours, or a range of 1 hour to 3 hours.

Optionally, the activated beads can be separated or washed. For example, the suspension can be centrifuged to pelletize the beads, and the solution can be separated from the pelletized beads. The pelletized beads can be resuspended in an aqueous solution. In an example, the aqueous solution includes a buffer, such as a PBS buffer or a TE buffer.

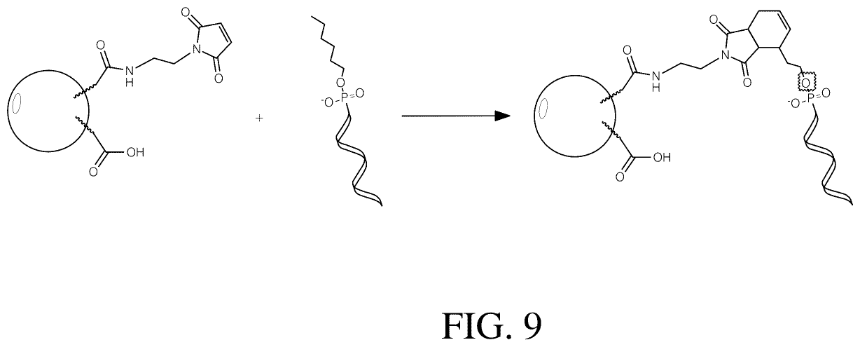

In an example, a diene modified oligonucleotide can be added to the suspension. The modified oligonucleotide can include a diene group or similar moiety at the 5′ end, for example, as illustrated in FIG. 9. In an example, the modified oligonucleotide can include a terminal moiety, such as a furan moiety, cyclopentadiene moiety, anthracene moiety, butadiene moiety, isoprene moiety, combinations thereof, or equivalents thereof. For example, the modified oligonucleotide includes a butadiene terminal moiety or an isoprene terminal moiety. In an example, the modified oligonucleotide includes a butadiene terminal moiety.

The terminal moiety can be connected to a phosphate group of an oligonucleotide through an intermediate structure, such as an alkyl group, aryl group, or glycol ether group. For example, the intermediate structure can include an alkyl group, such as an ethyl, propyl, butyl, or pentyl group. In an example, the diene modified oligonucleotide can include 1,3-hexadiene modified oligonucleotide.

The modified oligonucleotide can have between 10 and 50 nucleotides. For example, the modified oligonucleotide can have between 15 and 35 nucleotides, such as between 20 and 35 nucleotides.

The diene modified oligonucleotide can be added to a concentration in a range of 0.1 mM to 5 mM, such as a range of 0.5 mM to 3.5 mM or a range of 1.5 mM to 3.0 mM. In another example, the diene modified oligonucleotide can be added to an equivalent relative to the initial number of carboxy functional groups in a range of 1 eq to 50 eq, such as a range of 2 eq to 30 eq or a range of 5 eq to 20 eq.

The reaction can be held at a temperature in a range of 23° C. to 80° C., such as a range of 35° C. to 75° C. or a range of 40° C. to 70° C. The reaction can be held for a period in a range of 10 minutes to 70 hours, such as a range of 30 minutes to 60 hours, a range of 1 hour to 50 hours, or a range of 16 hours to 48 hours. As a result of the reaction, an oligonucleotide is secured to the beads.

In a further example, each of the DMTMM, amino-maleimide, and diene modified oligonucleotide can be added concurrently to the suspension. The reaction can be held at a temperature in a range of 23° C. to 80° C., such as a range of 35° C. to 75° C. or a range of 40° C. to 70° C. The reaction can be held for a period in a range of 10 minutes to 70 hours, such as a range of 30 minutes to 60 hours, a range of 1 hour to 50 hours, or a range of 16 hours to 48 hours.

### EXAMPLES

### Example 1

Carboxy functional beads (manufactured by Dynal) having an average size in a range of 0.35 micrometers to 1.2 micrometers and an initial carboxy functional group count in a range of 0.01 M COOH/bead to 8.59 M COOH/bead as conjugated.

To an aqueous suspension of the beads including PBS buffer and 50% NMP, DMTMM (50 eq.) and amino-maleimide (10 eq.) are added. DIPEA is added to a final concentration of 36 mM. The reaction occurs at room temperature for a period of 8 hours. Over 200K of the carboxy functional groups are activated.

Diene oligonucleotide is added at a concentration of 2.5 mM. The reaction proceeds at 40° C. for 8 hours, yielding beads with between 178K and 278K conjugated groups.

### Example 2

### Reagent and Sample Preparation

Set thermomixer to room temperature, 750 rpm, with the 2 mL tube insert. Remove hydrogel bead stock (water as the solvent) from 4° C. fridge and vortex the bottle for 2 min. Take out diene-Av4 oligo stock solution from freezer and kept on ice.

Make 0.5M DMTMM stock solution in water. Weight out 20 mg of DMTMM powder, add 145 μL of N-methyl-2-pyrrolidone (NMP). Vortex 30s for complete dissolution.

Make 0.5M N-(2-aminoethyl) maleimide hydrochloride (Maleimide HCl) stock solution in water. Weight out 20 mg of Maleimide powder, add 226 μL of water. Vortex 30s for complete dissolution.

Make 1.91M N-ethyldiisopropylamine (DIPEA) stock solution in water by diluting 10 μL of neat DIPEA with 20 μL of NMP. Neat DIPEA has limited solubility in pure water.

### Activation

With P1000 electronic pipettor, aspirate hydrogel bead stock, wiping off excess on the bead stock tube rim. Dispense 248.6 μL on the side wall of a labeled 2 mL centrifuge tubes. Put the tube back on ice.

With P1000 electronic pipette, aspirate PBS buffer (pH 6.5) and dispense 383.1 μL into the 2 mL centrifuge tube. Dispense the buffer on the side of the tube wall. Vortex for 5s and put the tube back on ice.

With P10 electronic pipette, aspirate DIPEA stock solution and dispense 4.5 μL into the 2 mL centrifuge tube. Dispense the solution on the side of the tube wall. Vortex for 5s and put the tube back on ice.

With P200 electronic pipettor, aspirate Maleimide stock solution, and dispense 27.3 μL into the 2 mL centrifuge tube. Dispense the solution on the side of the tube wall. Vortex for 5s and put the tube back on ice.

With P200 electronic pipettor, aspirate DMTMM stock solution, and dispense 136.5 μL into the 2 mL centrifuge tube. Dispense the solution on the side of the tube wall. Vortex for 5s and put the tube back on ice. The total volume of the activation reaction is 800 μL. The molar ratio of DMTMM:Maleimide:—COOH is 50:10:1. Incubate the mixture on the thermo-mixer at room temperature for 16 h.

### Conjugation

With P1000 electronic pipette, aspirate PBS buffer (pH 6.5) and dispense 400 μL into the 2 mL centrifuge tube. Dispense the buffer on the side of the tube wall.

With P1000 electronic pipette, aspirate diene-Av4 oligo stock solution (5 mM in PBS buffer) and dispense 400 μL into the 2 mL centrifuge tube. Dispense the buffer on the side of the tube wall. Vortex for 5s and put the tube back on the thermo-mixer.

Set the thermo-mixer temperature to 40° C., 750 rpm. Let the conjugation reaction carry out for 16 h.

### Workup

The sample tube is centrifuged at 250,000 rpm for 1 h to pellet the hydrogel beads. After the spin, supernatant is decanted, and 1 mL of water is added to the pellet. The sample tube is sonicated to resuspend the beads. Repeat twice to wash away non-reacted reagents. The hydrogel pellet is resuspended in low TE buffer for storage.

### Primer Load Characterization

The primer load is determined by FAM assay. Briefly, the conjugated hydrogel bead solution is diluted to 1M/uL. In a PCR tube, 45 μL of annealing buffer, 5 μL of the hydrogel bead solution, and 1 μL of FAM-Av4′ probe (100 μM) are mixed.

The PCR tube is loaded onto a thermo-cycler for one cycle of probe hybridization. The tube is heated to 97° C. for 2 min, then the temperature gradually drops to 37° C. and is kept for 2 min, then the tube is maintained at 4° C.

The hybridized sample is further washed with guava buffer to remove excess FAM-Av4′ probes, and the sample is measured by Guava Flow Cytometer to determine the primer load.

In a first aspect, a method is provided for conjugating oligonucleotides to bead supports. The method comprises adding 4-(4,6-dimethoxy-1,3,5-triazin-2-yl)-4-methyl-morpholinium to an aqueous suspension including bead supports having carboxy functional groups, adding amino-maleimide to the aqueous suspension, and adding diene modified oligonucleotide to the aqueous suspension.

In an example of the first aspect and above examples, the bead support includes a polyacrylamide polymer matrix.

In another example of the first aspect and above examples, the method further comprises adding a base when adding 4-(4,6-dimethoxy-1,3,5-triazin-2-yl)-4-methyl-morpholinium. In a further example of the first aspect and above examples, the base includes a hindered amine. In an additional example of the first aspect and above examples, the hindered amine includes diisopropylethylamine.

In another example of the first aspect and above examples, the method further includes incubating the suspension at a temperature in a range of 0° C. to 35° C. following adding 4-(4,6-dimethoxy-1,3,5-triazin-2-yl)-4-methyl-morpholinium.

In a further example of the first aspect and above examples, incubating includes incubating for a period in a range of 10 minutes to 100 hours.

In an additional example of the first aspect and above examples, adding 4-(4,6-dimethoxy-1,3,5-triazin-2-yl)-4-methyl-morpholinium includes adding to an equivalence ratio in a range of 1 eq. to 150 eq.

In another example of the first aspect and above examples, the method further comprises dissolving 4-(4,6-dimethoxy-1,3,5-triazin-2-yl)-4-methyl-morpholinium in a solvent prior to adding 4-(4,6-dimethoxy-1,3,5-triazin-2-yl)-4-methyl-morpholinium chloride.

In a further example of the first aspect and above examples, the solvent includes an amide, a urea, a carbonate, an ether, a sulfoxide, a sulfone, a hindered alcohol, or a combination thereof. In an additional example of the first aspect and above examples, the solvent includes dimethylformamide. In another example of the first aspect and above examples, the solvent includes n-methylpyrrolidone.

In an additional example of the first aspect and above examples, adding amino-maleimide includes adding amino-maleimide to an equivalent in a range of 1 eq. to 150 eq.

In a further example of the first aspect and above examples, the method further comprises wherein the amino-maleimide includes aminoalkyl maleimide. In an additional example of the first aspect and above examples, the aminoalkyl maleimide includes aminoethyl maleimide.

In another example of the first aspect and above examples, the method further comprises incubating at a temperature in a range of 0° C. to 35° C. following adding amino-maleimide to the aqueous suspension. In a further example of the first aspect and above examples, incubating included incubating for a period in a range of 10 minutes to 100 hours.

In an additional example of the first aspect and above examples, the diene modified oligonucleotide includes 1,3 hexadiene modified oligonucleotide.

In another example of the first aspect and above examples, the method further comprises incubating at a temperature in a range of 23° C. to 80° C. following adding diene modified oligonucleotide. In a further example of the first aspect and above examples, incubating includes incubating for a period in a range of 10 minutes to 70 hours.

In an additional example of the first aspect and above examples, the diene modified oligonucleotide is added to a concentration in a range of 0.1 mM to 5 mM. In another example of the first aspect and above examples, the diene modified oligonucleotide is added to an equivalent in a range of 1 eq. to 50 eq.

In a further example of the first aspect and above examples, adding 4-(4,6-dimethoxy-1,3,5-triazin-2-yl)-4-methyl-morpholinium and adding amino-maleimide occur concurrently, the method further including incubating at a temperature in a range of 0° C. to 35° C. for a period in a range of 10 minutes to 100 hours following adding 4-(4,6-dimethoxy-1,3,5-triazin-2-yl)-4-methyl-morpholinium chloride and amino-maleimide.

In a second aspect, a method is provided for conjugating oligonucleotides to bead supports. The method comprises adding 4-(4,6-dimethoxy-1,3,5-triazin-2-yl)-4-methyl-morpholinium to an aqueous suspension including bead supports having carboxy functional groups, adding an amino modified maleimide to the aqueous suspension, and adding modified biomolecule to the aqueous suspension, the modified biomolecule including a terminal group including a furan moiety, cyclopentadiene moiety, anthracene moiety, butadiene moiety, isoprene moiety, or combinations thereof.

In an example of the second aspect, the amino modified maleimide includes aminoalkyl maleimide or aminophenyl maleimide.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A method for conjugating oligonucleotides to bead supports, the method comprising:
adding 4-(4,6-dimethoxy-1,3,5-triazin-2-yl)-4-methyl-morpholinium to an aqueous suspension including bread supports having carboxy functional groups;
adding amino-maleimide to the aqueous suspension; and
adding diene modified oligonucleotide to the aqueous suspension.

2. The method of claim 1, wherein the bead support includes a polyacrylamide polymer matrix.

3. The method of claim 1, further comprising adding a base when adding 4-(4,6-dimethoxy-1,3,5-triazin-2-yl)-4-methyl-morpholinium.

4. The method of claim 3, wherein the base includes a hindered amine.

5. The method of claim 4, wherein the hindered amine includes diisopropylethylamine.

6. The method of claim 1, further including incubating the suspension at a temperature in a range of 0° C. to 35° C. following adding 4-(4,6-dimethoxy-1,3,5-triazin-2-yl)-4-methyl-morpholinium.

7. The method of claim 6, wherein incubating includes incubating for a period in a range of 10 minutes to 100 hours.

8. The method of claim 1, wherein adding 4-(4,6-dimethoxy-1,3,5-triazin-2-yl)-4-methyl-morpholinium includes adding to an equivalence ratio in a range of 1 eq. to 150 eq.

9. The method of claim 1, further comprising dissolving 4-(4,6-dimethoxy-1,3,5-triazin-2-yl)-4-methyl-morpholinium in a solvent prior to adding 4-(4,6-dimethoxy-1,3,5-triazin-2-yl)-4-methyl-morpholinium chloride.

10. The method of claim 9, wherein the solvent includes an amide, a urea, a carbonate, an ether, a sulfoxide, a sulfone, a hindered alcohol, or a combination thereof.

11. (canceled)

12. (canceled)

13. The method of claim 1, wherein adding amino-maleimide includes adding amino-maleimide to an equivalent in a range of 1 eq. to 150 eq.

14. The method of claim 1, further comprising wherein the amino-maleimide includes aminoalkyl maleimide.

15. (canceled)

16. The method of claim 1, further comprising incubating at a temperature in a range of 0° C. to 35° C. following adding amino-maleimide to the aqueous suspension.

17. The method of claim 16, wherein incubating included incubating for a period in a range of 10 minutes to 100 hours.

18. The method of claim 1, wherein the diene modified oligonucleotide includes 1,3 hexadiene modified oligonucleotide.

19. The method of claim 1, further comprising incubating at a temperature in a range of 23° C. to 80° C. following adding diene modified oligonucleotide.

20. The method of claim 19, wherein incubating includes incubating for a period in a range of 10 minutes to 70 hours.

21. The method of claim 1, wherein the diene modified oligonucleotide is added to a concentration in a range of 0.1 mM to 5 mM.

22. The method of claim 1, wherein the diene modified oligonucleotide is added to an equivalent in a range of 1 eq to 50 eq.

23. The method of claim 1, wherein adding 4-(4,6-dimethoxy-1,3,5-triazin-2-yl)-4-methyl-morpholinium and adding amino-maleimide occur concurrently, the method further including incubating at a temperature in a range of 0° C. to 35° C. for a period in a range of 10 minutes to 100 hours following adding 4-(4,6-dimethoxy-1,3,5-triazin-2-yl)-4-methyl-morpholinium chloride and amino-maleimide.

24.-25. (canceled)
